(19) United States
(12) Patent Application Publication
(10) Pub. No.: US 2011/0262466 A1
(43) Pub. Date: Oct. 27, 2011

(54) COMPOSITIONS CONTAINING THROMBOMODULIN DOMAINS AND USES THEREOF

(75) Inventors: Vladimir R. Muzykantov, Warwick, PA (US); Bi-Sen Ding, New York, NY (US); Douglas B. Cines, Wynnewood, PA (US); Claudia Gottstein, Carpinteria, CA (US); Steven M. Albelda, Philadelphia, PA (US)

(73) Assignee: The Trustees of the University of Pennsylvania, Philadelphia, PA (US)

(21) Appl. No.: 13/124,295

(22) PCT Filed: Oct. 16, 2009
(86) PCT No.: PCT/US2009/060938

§ 371 Date: Jul. 11, 2011

Related U.S. Application Data
(60) Provisional application No. 61/105,956, filed on Oct. 16, 2008.

(51) Int. Cl. A61K 39/395; A61P 7/02; A61P 7/10; A61P 29/00; A61P 9/10; C07K 19/00; A61P 11/00

(52) U.S. Cl. 424/178.1; 530/391.7

(57) ABSTRACT

Compositions are provided comprising a thrombomodulin domain linked to a targeting moiety that binds to a determinant on the surface of a target endothelial cell or red blood cell, wherein the thrombomodulin domain may be the extracellular domain, the N-terminal lectin-like domain, or an epidermal growth factor (EGF)-like domain. The targeting moiety may be a single chain antigen-binding domain (scFv), and the targeting moiety and thrombomodulin domain of the composition may be linked as a continuous polypeptide chain. Methods of delivery and use of a composition described herein are provided, as well as methods of treating or preventing thrombosis, inflammation, tissue ischemia, sepsis, acute lung injury (ALI), acute myocardial infarction (AMI), ischemic stroke, cerebrovascular disease, pulmonary embolism, or ischemic peripheral vascular disease is provided.

### STATEMENT REGARDING FEDERALLY SPONSORED RESEARCH OR DEVELOPMENT

This work was sponsored by grants from the National Institutes of Health, CA-83121, HL-71174, HL-71175, HL-76206, HL-76406, HL-82545, HL-79063, HL-90697, HL-91950, and the Department of Defense, PR-12262.

### BACKGROUND OF THE INVENTION

Thrombomodulin (TM) is an integral membrane protein expressed on the surface of endothelial cells. Human TM consists of a single polypeptide chain with 5 distinct domains: an NH₂-terminal lectin-like region designated D1, which comprises Ala¹ through Asp²²⁶; a domain with 6 epidermal growth factor (EGF)-like domains joined by small interdomain peptides (Cys²²⁷ through Cys⁴⁶²) designated D2; an O-glycosylation site-rich/serine/threonine rich domain (Asp⁴⁶³ through Ser⁴⁹⁷) designated D3; a transmembrane domain consisting of (Gly⁴⁹⁸ through Leu⁵²¹) designated D4; and a cytoplasmic tail domain (Arg⁵²²through Leu⁵⁵⁷) designated D5. [Shi, et al., Evidence of Human Thrombomodulin Domain as a Novel Angiogenic Factor, Circulation 2005; 111:1627-1636 (Mar. 28, 2005).] The six subdomains of the D2 domain are EGF1 (227-262), EGF2 (270-305), EGF3 (311-344), EGF4 (351-386), EGF5 (390-407), and EGF6 (427-462). [Wang, et al., Elements of the Primary Structure of Thrombomodulin Required for Efficient Thrombin-activatable Fibrinolysis Inhibitor Activation, J. Biol. Chem., 275(30): 22942-22947 (Jul. 28, 2000).]

Thrombomodulin exerts direct effects in the endothelium, such as binding to HMGB1, a proinflammatory mediator that contributes to lethality in sepsis and acute lung injury (ALI). [Abeyama, et al., The N-terminal domain of thrombomodulin sequesters high-mobility group-B1 protein, a novel antiinflammatory mechanism, J. Clin. Invest., 115:1627-1274 (May 2005).] In addition to direct effects in the endothelium, TM binds thrombin, a procoagulant enzyme which converts soluble fibrinogen into fibrin, with high affinity to form a 1:1 complex. This binding blocks thrombin's procoagulant and proinflammatory effects. Further, TM-bound thrombin is a potent activator of protein C. Activated protein C (APC), together with its co-factor vitamin K-dependent Protein S, catalyzes the proteolytic degradation of the membrane-bound thrombin-activated forms of coagulation factors V and VIII (Va and VIIIa). Further, APC interaction with the endothelial protein C receptor triggers antiinflammatory responses. Thus, thrombomodulin is a key component of thrombotic and inflammatory processes. However, thrombomodulin's diverse effects and difficulty in effectively delivering agents to the endothelium have limited its therapeutic usefulness.

Given thrombomodulin has both antithrombotic and antiinflammatory effects, administration as an antiinflammatory agent provides a risk of bleeding (via its antithrombotic effect). However, where antithrombotic effects are desired, administration provides potentially undesirable antiinflammatory effects.

Further, effectively overcoming inefficiency (e.g., blood clearance) and achieving targeted effectiveness of an agent is also a concern. For example, high doses of antithrombotic agents, such as plasminogen activators (PAs) or activated protein C (APC), are required in treating or preventing thrombosis (pathological intravascular occlusion by clots, which can cause tissue ischemia and damage leading to acute myocardial infarction (AMI), ischemic stroke, pulmonary embolism and ischemic peripheral vascular disease, among other conditions). However, high doses may cause bleeding by disruption of hemostatic mural clots, including bleeding into the central nervous system (CNS) causing neuronal toxicity and inflammation in the brain.

In addition to problems of drug clearance (and related undesirable dosage side effects), synthesis, expression and activity of native endothelial thrombomodulin are suppressed in a number of disorders. In particular, suppression has been identified in inflammatory and thrombotic conditions of the pulmonary vasculature including acute lung injury, sepsis and ischemia/reperfusion (I/R), and hyperoxia.

What is needed are compositions and methods that enable thrombomodulin to be used for its therapeutic advantages without incurring its undesirable side effects.

### SUMMARY OF THE INVENTION

The above-stated need in the art is met by providing compositions containing domains of thrombomodulin specifically targeted to the surface of the endothelium or to a red blood cell.

In one aspect, a composition is provided comprising a thrombomodulin domain linked to a targeting moiety that binds to a surface determinant of a target cell. In a further embodiment, the thrombomodulin domain is the extracellular domain of thrombomodulin. In yet another embodiment, the thrombomodulin domain is the N-terminal lectin-like domain of thrombomodulin. In still another embodiment, the thrombomodulin domain is an epidermal growth factor (EGF)-like domain of thrombomodulin. In a further embodiment, the EGF-like domain is EGF 4-6.

In another aspect, the targeting moiety of the composition is a polypeptide chain, such as a single chain antigen-binding domain (scFv).

In yet another aspect, the targeting moiety and thrombomodulin domain of the composition are linked as a continuous polypeptide chain. In another embodiment, the targeting moiety and thrombomodulin domain are chemically cross-linked.

In another aspect, the targeting moiety of the composition binds to a surface determinant on the surface of vascular endothelium, represented by a glycoprotein such as GP-90, a phospholipid such as PS, or a cell adhesion molecule. In another embodiment, the targeting moiety binds to a cell adhesion molecule on a luminal surface of vascular endothelium, such as PECAM-1, ICAM-1, or VCAM-1. In another embodiment, the targeting moiety binds a surface determinant expressed on the surface of a red blood cell. In another embodiment, the targeting moiety binds a surface determinant expressed on the surface of a red blood cell at a density greater than 5,000 copies per red blood cell. In still a further embodiment, the RBC surface determinant is a glycophorin A (in primates) or glycophorin A-associated proteins (in non-primates).

In still another aspect, a pharmaceutical composition is provided comprising a composition described herein and a pharmaceutically acceptable carrier.

In other embodiments, methods of delivery and use of a composition described herein are provided. In one embodiment, a method of delivering a thrombomodulin domain to a luminal surface of vascular endothelium is provided. In another embodiment, a method of delivering a thrombomodulin domain to the surface of a red blood cell is provided. In still another embodiment, a method of treating, inhibiting, or preventing thrombosis, tissue ischemia, acute myocardial infarction (AMI), non-ST segment elevated AMI, deep vein thrombosis, hyperoxic injury, transient ischemic attack (TIA), ischemic stroke, cerebrovascular disease, disseminated intravascular coagulation (DIC), pulmonary embolism, ischemic peripheral vascular disease, inflammation, pulmonary edema, sepsis, acute lung injury (ALI), acute respiratory distress syndrome (ARDS), or aseptic systemic inflammation is provided. Medicaments for use in treating or preventing these conditions are also provided.

Other aspects and advantages of these methods and compositions are discussed in detail below.

### BRIEF DESCRIPTION OF THE DRAWINGS

FIG. 1 illustrates the molecular design of an anti-PECAM scFv/thrombomodulin (TM) extracellular domain composition. The extracellular domain of mouse TM was fused with the anti-PECAM-1 scFv.

FIG. 2 illustrates the functional properties of scFv/TM in vitro. FIG. 2A is a bar graph showing activation of protein C indicated by cleavage of its substrate by scFv/TM. scFv/TM induced dose-dependent activation of protein C in the presence of thrombin. FIG. 2B is a bar graph showing that scFv/TM demonstrated comparable protein C-activating cofactor ability to sTM. FIG. 2C is a graph showing that scFv/TM binds mouse PECAM-1, revealed by anti-TM ELISA. FIG. 2D is a bar graph showing activation of protein C by PECAM-associated scFv/TM. PECAM-coated wells pre-incubated with scFv/TM, but not soluble TM (sTM), generated activated Protein C (APC) activity upon thrombin addition. Phosphate buffered saline with bovine serum albumin (PBS/BSA) was used as a control.

FIG. 3A provides a plate from an agglutination (aggregation) assay, reflecting binding of HMGB1 to TM (of the Ter119scFv/TM composition). Polyclonal anti-HMGB1 yields agglutination only in Ter119-TM loaded RBCs. FIG. 3B is a Western Blot (immunoblot-IB) confirming the agglutination assay.

FIG. 4 is a bar graph showing the functional activity of a composition comprising an anti-glycophorin A associated protein (mouse RBC) scFv derived from parental rat MAb Ter119, and an extracellular domain of mouse thrombomodulin (Ter119-TM). Mouse and human RBCs were incubated in serum free medium from induced and non-induced S2 cells transfected with a plasmid encoding Ter119-TM. RBC were washed and incubated with protein C in the presence or absence of thrombin. The experiment demonstrated that only mRBC loaded with Ter119-TM in the presence of thrombin cause protein C activation thus confirming both antigen binding and functional activity of Ter119-TM. The columns are marked as induced murine RBC (mRBC 1) with (+) or without (−) thrombin; non-induced murine RBC (mRBC) with (+) or without (−) thrombin; induced human RBC (hRBC I) with (+) or without (−) thrombin; non-induced human RBC (hRBC) with (+) or without (−) thrombin. The data is obtained with known loading of RBC by scFv-TM (˜25,000 molecules per mRBC).

FIG. 5A shows distribution of ⁵¹Cr-labeled and Ter119-TM-¹²⁵I loaded mouse RBCs at 1 h, 3 h, and 6 h, for control RBC, ⁵¹Cr-labeled RBCs loaded with Ter119-TM, and mouse RBCs loaded with Ter119-TM-¹²⁵I . FIG. 5B reflects corresponding blood component distributions (RBC vs. plasma).

FIG. 6A reflects Ter119-TM distribution in blood and organs at time points through 48 hours (left to right 0.5 h (no results shown), 1 h, 3 h, 6 h, 24 h, and 48 h). FIG. 6B reflects blood component distributions (RBC vs. plasma) at time points (left to right) 0.5 h, 1 h, 3 h, 6 h, 24 h, and 48 h.

FIG. 7A reflects Ter119-TM distribution in blood and organs, when delivered by intraperitoneal (IP) injection, at time points 1 h and 3 h. FIG. 7(B) reflects blood component distributions (RBC vs. plasma) at time points 1 h and 3 h.

FIG. 8 illustrates mouse acute inflammatory lung injury. FIG. 8A is a bar graph showing that mice given an intratracheal injection of LPS followed by exposure to 98% O₂ (LPS/hyperoxia) showed marked reduction in lung TM vs. mice administered a sham composition. FIG. 8B is a bar graph showing that mice treated similarly as in FIG. 8A showed an increase in cytokine high mobility group-B1 (HMGB1) as compared to mice administered a sham composition. FIG. 8C is a graph reflecting interaction between scFv/TM () and HMGB1, and scFv uPA-T (∘) and HMGB1. scFv/TM bound HMGB1, whereas a thrombin-activated urokinase plasminogen activator (uPA) fused with the same scFv moiety (seFv/uPA-T) did not. The asterisk represents statistical difference between two groups (p<0.05).

FIG. 9 illustrates scFv/TM attenuation of inflammation. FIG. 9A provides an overview of the experimental design. FIG. 9B is a bar graph illustrating the effect of scFv/TM on myeloperoxidase (MPO) level in the inflamed lungs compared to that of sTM and PBS as a control. FIGS. 9C and 9D are graphs illustrating scFv/TM alleviation of the expression of inflammatory cell adhesion molecules ICAM-1 (9C) and VCAM-1 (9D), as compared to a PBS control and sTM. FIG. 9E is a graph that illustrates inhibition of NF-κB activation by scFv/TM following acute lung injury (ALI), as contrasted with sTM. Dashed lines show levels in sham-operated animals. The asterisk(s) represents statistical difference between two groups (p<0.05).

FIG. 10 illustrates prevention of lung edema by scFv/TM. FIG. 10A is a bar graph that shows scFv/TM post-injury injection attenuates the increased lung wet to dry ratio as compared with sTM and PBS FIG. 10B is a bar graph that shows the pathological elevation of lung vascular permeability by post-injury injection of scFv/TM via diminished Evans blue extravasation, as compared with sTM and PBS. FIG. 10C is a bar graph that shows that scFv/TM does not prolong the tail bleeding time as compared with PBS and APC. Dashed lines show levels in sham-operated animals. Asterisks denote statistical significance of differences with control groups. The asterisk(s) represents statistical difference between two groups (p<0.05).

### DETAILED DESCRIPTION OF THE INVENTION

Compositions are provided comprising a thrombomodulin domain linked to a targeting moiety that binds to a determinant on the surface of a target cell. In one embodiment, the targeting moiety binds a cell adhesion molecule on a surface of vascular endothelium. As used herein, the terms “vascular endothelium” and “endothelium” are used herein interchangeably, and are intended to have the same meaning. In another embodiment the targeting moiety binds a determinant expressed on the surface of a red blood cell. In other embodiments, pharmaceutical compositions are provided as well as methods of delivering a thrombomodulin domain. Methods of treating or preventing pathological conditions are provided, as well as use of the compositions described in preparing a medicament therefore.

### I. THROMBOMODULIN DOMAINS

As described above, thrombomodulin is composed of several domains (also referred to as subdomains). The amino acid sequence of human thrombomodulin is provided in Genbank® Accession No. AAM03232, and is provided herein as SEQ ID NO: 2. A nucleotide sequence encoding human thrombomodulin is provided in Genbank® Accession No. AF495471, and is provided herein as SEQ ID NO: 1. The mature sequence (excluding the signal peptide which extends from amino acids 1-18, inclusive) is referenced in Shi, et al., Circulation 2005 (referenced in the Background). The examples to this specification utilize and reference the mouse thrombomodulin domains (for use in the mouse models), the amino acid sequence for which is provided in Genbank® Accession No. NP^{—}033404, and is provided herein as SEQ ID NO: 3. However, one of skill in the art will readily understand that the corresponding human thrombomodulin domains (and sequences therefore) will be useful in the compositions described herein. These domains (with corresponding human sequences) are described as follows.

In one embodiment, the thrombomodulin domain utilized in a composition described herein is the extracellular domain of human thrombomodulin. This domain extends from amino acids Ala¹-Ser⁴⁹⁷ (Ala¹⁹-Ser⁵¹⁵ of SEQ ID NO: 2, i.e., including signal sequence). In another embodiment, the thrombomodulin domain is the lectin-like domain (aa 19-244 of SEQ ID NO: 2; TM^{Lec}).

In still another embodiment, the thrombomodulin domain, is an epidermal growth factor (EGF)-like domain. In one embodiment, the composition contains the full EGF-like domain, i.e., EGF1-6 (aa 245-480 of SEQ ID NO: 2; TM^{EGFs}). In another embodiment, the composition contains only selected EGF-like domains. In a further embodiment, the composition contains only the EGF3-6 domain (aa 329-480 of SEQ ID NO: 2). In a yet another embodiment, the composition contains only the EGF4-6 domain (aa 369-480 of SEQ ID NO: 2; TM^{EGF4-6}). In still another embodiment, the composition contains only the EGF5-6 domain (aa 408-480 of SEQ ID NO: 2).

In still other embodiments, the thrombomodulin domain is provided in a latent form, which exerts functional activity selectively in the therapeutic site. In one embodiment, this feature is provided by dependence of TM functions on its contact with pathological mediators including cytokines (such as HMGB-1) and proteases (such as thrombin). In another embodiment, the insertion of an activation site permits the thrombomodulin domain to be specifically released or activated at a pathological site. In a specific embodiment, such pathological sites are sites of coagulation or inflammation. In one embodiment, an activation site is interposed between the targeting moiety and the TM domain. In one embodiment, the activation site is a protease cleavage site, such as cleavage sites for proteases from leukocytes including but not limited to cathepsin G or elastases, proteases degrading extracellular matrix including but not limited to collagenases or metalloproteinases, and proteases involved in plasma cascades of coagulation, complement or kinins. Molecules that are generated during the natural coagulation process and that can serve as activating molecules in this sense include, but are not limited to, coagulation factors such as factor Xa, plasminogen, thrombomodulin-activatable fibrinolysis inhibitor (TAFI) and thrombin. In one embodiment, a composition containing only EGF-like domains (excluding the lectin-like domain) is provided as a latent drug activated by a molecule involved in the coagulation process. In a further embodiment, a composition containing only EGF3-6, 4-6, or 5-6 is provided as a latent drug activated by a molecule involved in the coagulation process.

In other embodiments, a composition containing only a lectin-like domain (excluding the EGF-like domain, or subdomains thereof) is provided as a latent drug activated by a molecule involved in the antiinflammatory process. In one embodiment, the activation molecule is a cytokine. In other embodiments, the activation molecule is a pro-inflammatory cytokine, such as IL1-alpha, IL1-beta, IL6, and TNF-alpha, LIF, IFN-gamma, OSM, CNTF, TGF-beta, GM-CSF, IL11, IL12, IL17, IL18, and IL8. In yet another embodiment, the activation molecule is myeloperoxidase, or lung myeloperoxidase. Still other inflammatory and coagulation (thrombotic) mediators as known to one of skill in the art may be the activation molecule.

### II. TARGETING MOIETY AND TARGET SURFACE DETERMINANT

A. Moiety

In one embodiment, the targeting moiety is a ligand that binds specifically to a target surface determinant expressed on a target cell, e.g., on the surface of the endothelium or on the surface of a red blood cell. In a further embodiment, the ligand is a polypeptide. In still a further embodiment, the polypeptide is an antibody or fragment thereof.

In one embodiment, the targeting moiety is an amino acid sequence of between about 50 kD MW and about 100 kD MW, which binds to the surface determinant. In a further embodiment, the targeting moiety is an amino acid sequence of between about 60 kD MW and about 100 kD MW. In still a further embodiment, the targeting moiety is an amino acid sequence of between about 70 kD MW and about 100 10 MW.

In a further embodiment, the targeting moiety is monovalent, i.e., it binds to a single binding site on a single target cell, e.g., a monoclonal antibody. Such monovalent targeting moieties avoid cross-linking of binding determinants, thereby avoiding potentially harmful cell membrane modification and cell aggregation. Other targeting moieties include a humanized antibody, a synthetic antibody, a heavy chain antibody, and a biologically active fragment of an antibody, such as a Fab, F(ab′)₂, or an scFv.

In another embodiment, the targeting moiety is a single chain antigen-binding domain (scFv) of a monoclonal antibody. scFvs may be generated conventionally, e.g., by the method of Spitzer, et al. (Mol. Immunol. 2003, 40:911-919). Total RNA of a hybridoma cell line is isolated (e.g., by RNeasy, Qiagen, Velencia, Calif.), followed by reverse transcription, e.g., using the SMART™ technology (Clontech, Palo Alto, Calif.) employing known primers (e.g., those of Dübel, et al. (J. Immunol. Methods 1994, 175:89-95)). The resulting heavy (V_{H}) and light (V_{L}) chain variable cDNA fragments are then subcloned into a suitable plasmid, e.g., pCR®2.1-TOPO® (Invitrogen, Carlsbad, Calif.). The materials utilized are not a limitation of these embodiments. The V_{H} and V_{L} chains generated are combined with a suitable linker (such as those described herein), resulting in the desired scFv. Since scFvs are monovalent, they are not anticipated to cross-link target cell determinants, such as RBC determinants, which is the main safety concern for using most monoclonal antibodies.

Other targeting moieties include polypeptide sequences prepared by phage display or other known methods which are capable of binding to a determinant and linking to the selected thrombomodulin domain. One of skill in the art provided with the teachings of this specification and publically available information can readily design a polypeptide useful in the compositions described herein.

Suitable production techniques are well known to those of skill in the art. See, e.g., Sambrook et al, Molecular Cloning: A Laboratory Manual, Cold Spring Harbor Press (Cold Spring Harbor, N.Y.). Alternatively, peptides can also be synthesized by the well known solid phase peptide synthesis methods (Merrifield, J. Am. Chem. Soc., 85:2149 (1962); Stewart and Young, Solid Phase Peptide Synthesis (Freeman, San Francisco, 1969) pp. 27-62). Polymerase chain reaction (PCR) and related techniques are described in Derbyshire, et al. (Immunochemistry 1: A practical approach. M. Turner, A. Johnston eds., Oxford University Press 1997, e.g., at pp. 239-273). Plasmids useful herein have been described in Derbyshire, et al. (cited above), as well as Gottstein, et al. (Biotechniques 30: 190-200, 2001). Cloning techniques are also described in these and other suitable production methods are within the knowledge of those of skill in the art and are not a limitation of the compositions and methods described herein. Generation of recombinant proteins provides flexibility in design, rapid production, large-scale production and uniform composition. Biological means include purification from a biological source, recombinant synthesis and in vitro translation systems, using methods well known in the art.

B. Target Determinant

The determinant which the targeting moiety of the composition binds is any determinant expressed on a cell to which the thrombomodulin domain is desired to be delivered. In one aspect, the target cell is vascular endothelium, i.e., the inner layer of cells (endothelial cells) lining the luminal surface of the blood vessels. In another aspect, the determinant is located on the surface of a red blood cell.

Endothelium Surface Targets

In one embodiment, the targeting moiety specifically binds to an endothelial cell surface determinant. In a further embodiment, the determinant is stably expressed or up-regulated in thrombosis and/or inflammation. In a further embodiment, the determinant permits a composition as described herein to reside for a relatively prolonged time on the luminal surface of endothelial cells. In one embodiment, the determinant is a cell adhesion molecule (CAM). In a further embodiment, the determinant is Platelet-Endothelial Cell Adhesion Molecule-1 (PECAM-1). In another embodiment, the determinant is Intercellular Adhesion Molecule-1 (ICAM-1). In still a further embodiment, the determinant is Vascular Cell Adhesion Molecule-1 (VCAM-1). In yet another embodiment, the determinant is mucosal vascular adressin cell adhesion molecule (MAdCAM). Still other cell adhesion molecules and other endothelial cell surface determinants are known to one of skill in the art, and may be targeted by the compositions described herein.

Red Blood Cell Targets

In one embodiment of a red blood cell targeting moiety, the cell surface determinant to which the ligand binds is expressed across the human population at a density greater than 5,000 molecules per red blood cell. In another embodiment, the determinant is expressed across the human population at a density greater than 10,000 molecules per red blood cell. In another embodiment, the determinant is expressed across the human population at a density greater than 20,000 molecules per red blood cell. In still another embodiment, the determinant is expressed across the human population at a density greater than 50,000 molecules per red blood cell. In another embodiment, the determinant is expressed on the red blood cell at greater than 100,000 molecules per red blood cell. In another embodiment, the determinant is expressed on the red blood cell at greater than 500,000 molecules per red blood cell. In still further embodiments, the determinant is expressed on the surface of a red blood cell at a density greater than 1,000,000 molecules per red blood cell. In still further embodiments, the determinant is expressed on the surface of a red blood cell at a density greater than 2,000,000 molecules per red blood cell.

In a specific embodiment, the determinant is glycophorin A (GPA). In non-primates, the determinant is a glycophorin A-associated protein. Unless otherwise indicated, glycophorin A and glycophorin A-associated protein are used interchangeably, and the applicable glycophorin A associated protein (e.g. GPA or Ter119 (mouse)) would be understood/selected by one of skill in the art. In another embodiment, the determinant is an ABO blood group antigen. In another embodiment, the determinant is the Rhesus factor antigen. In another embodiment, the determinant is RBC band 3 antigen. Still other appropriate targeting determinants meeting the above density requirements may be selected by one of ordinary skill in the art.

In another embodiment, the determinant is not a specific site for specific functioning of the target cell. For example, a desirable RBC determinant is not a site necessary for recognition by host defense cells that clear microscopic objects from the surface of a red blood cell without damage to the red blood cell. Both the GPA and ABO blood group antigens meet this requirement.

### III. LINKAGE

The targeting moiety that binds to a determinant on the surface of a target cell may be linked to a thrombomodulin domain by any conventional means known in the art. In one embodiment, the targeting moiety is linked to a thrombomodulin domain by cross-linking a biotinylated thrombomodulin domain to a biotinylated target cell via streptavidin. [See, e.g., Streptavidin-mediated coupling of therapeutic proteins to the carrier erythrocytes. “Erythrocyte engineering for drug delivery and targeting”, M. Magnani, Ed., Kluwer Academic/Plenum Publishers, New York, Chapter 4, pages 37-67. See also U.S. Pat. No. 7,172,760.] Other known forms of chemical cross-linkage between the targeting moiety and the TM domain can also be used, e.g., by covalent or non-covalent linkage. In one embodiment, the cross-linkage is via covalent bond. In another embodiment, the cross-linkage is via non-covalent bond. However, other affinity binding pairs may be utilized, as known in the art. Still other conjugation chemistries known in the art are contemplated and may be used in embodiments herein, including but not limited to activated PEG-, maleimide or succinimide ester based cross-linkers or SATA-SMCC bifunctional cross-linkers, among other linkages known to those of skill in the art. Moreover, genetic engineering allows the design and synthesis of targeted pro-drugs which can be cleaved and thereby activated or released locally by pathophysiologically relevant enzymes that are generated at the site of a disorder or disease that cannot be attained using chemical conjugation.

In another embodiment, a targeting moiety is linked to a thrombomodulin domain as a continuous polypeptide chain via a ‘linker’. As noted above with respect to scFv targeting moieties, linkers may also be utilized to join variable heavy and variable light chain fragments. A linker as used herein refers to a chain of as short as about 1 amino acid to as long as about 100 amino acids, or longer. In a further embodiment, the linker is 3, 4, 5, 6, 7, 8, 9, 10, 11, 12, 13, 14, 15, 16, 17, 18, 19, or 20 amino acids in length. In one embodiment, the linker is 13 amino acids in length.

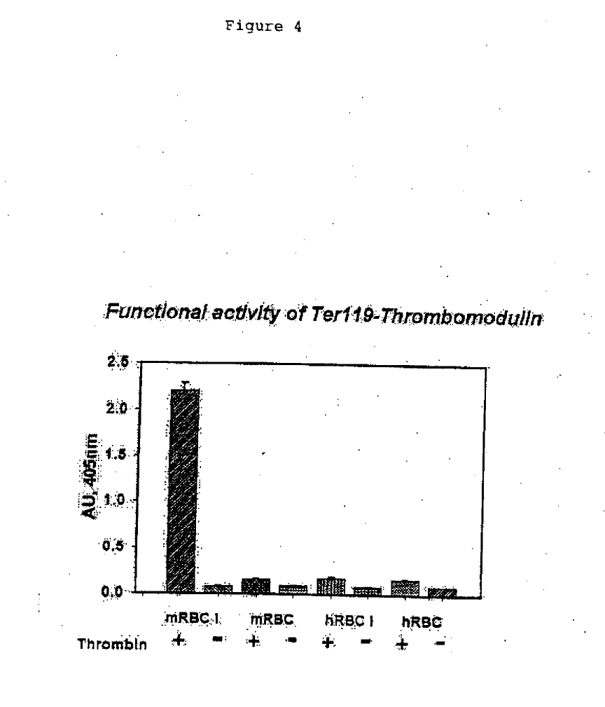

In one embodiment, the linker is -Ser-Ser-Ser-Ser-Gly-Ser-Ser-Ser-Ser-Gly-Ala-Ala-Ala-(SEQ ID NO: 4), i.e., “(S₄G)₂AAA” (See, e.g., FIG. 4). In another embodiment, the linker is (G₄S)₃, i.e., -Gly-Gly-Gly-Gly-S-Gly-Gly-Gly-Gly-S-Gly-Gly-Gly-Gly-S-(SEQ ID NO: 5)(Böldicke, et al. (Stem Cells 2001, 19:24-36)). However, as will be understood by one of skill in the art, other linkers can be utilized.

Since the thrombomodulin domain is bound to its target and therefore immobilized, diffusion capability is inhibited in all targeted approaches. Accordingly, in some embodiments, a cleavage site is incorporated into the linker between the targeting moiety and the thrombomodulin domain, which is cleaved by a protease upon initiation of coagulation or inflammation. This serves to overcome an impaired capability of the targeted molecules to diffuse e.g., into a clot. Such cleavage sites may occur naturally or may be engineered at the respective site.

In one embodiment, the cleavage site is a thrombin cleavage site. In a further embodiment, the thrombin cleavage site is Met-Tyr-Arg-Gly-Asn (SEQ ID NO: 6). In one embodiment, the cleavage site is specific for a protease occurring during coagulation (including but not limited to serine proteases of the blood coagulation system). For example, anti-PECAM scFv contains a natural short sequence of amino acids that form a thrombin-specific cleavage site, thus providing an ideal mechanism for local release and maximal activity of endothelium-bound drug in the site of active thrombosis. Thus, in one embodiment a composition of the present invention comprises an anti-PECAM scFv portion possessing a natural thrombin cleavage site, providing site- and time-specific liberation of a thrombomodulin domain in the site of active thrombosis. In another embodiment, the composition contains a cleavage site for cleavage by a molecule involved in an inflammatory process. In further embodiments, the molecule is a pro-inflammatory cytokine, such as IL1-alpha, IL1-beta, IL6, and TNF-alpha, LIF, IFN-gamma, OSM, CNTF, TGF-beta, GM-CSF, IL8, IL11, IL12, IL17, and IL18. In yet another embodiment, the molecule is myeloperoxidase or lung myeloperoxidase. Still other inflammatory and coagulation (thrombotic) mediators may be selected by one of skill in the art for generation of an appropriate cleavage site.

### IV. EXEMPLARY COMPOSITIONS

In one embodiment, a composition is provided comprising a thrombomodulin domain linked to an scFv that binds an determinant on the surface of an endothelial cell. In a further embodiment, the determinant is on the surface of vascular endothelium. In a further embodiment, the molecule is a cell adhesion molecule. In still a further embodiment, the cell adhesion molecule is ICAM-1, PECAM-1, or VCAM-1. In one embodiment, a composition comprises the extracellular domain of thrombomodulin, bound to a scFv that binds PECAM-1. In another embodiment, a composition comprises the extracellular domain of thrombomodulin, bound to an scFv that binds PECAM-1, ICAM-1, or VCAM-1. In a further embodiment, a composition binds PECAM-1. In a further embodiment, a composition comprises the lectin-like domain of thrombomodulin, bound to an scFv that binds PECAM-1. In still a further embodiment, a composition comprises an scFv that binds PECAM-1, bound to an EGF-like domain of thrombomodulin. In other embodiments, the EGF-like domain is EGF3-6, EGF4-6, or EGF5-6. In still other embodiments, the scFv of these compositions binds ICAM-1 rather than PECAM-1. In other embodiments, the scFv of these compositions binds VCAM-1 rather than PECAM-1. In another embodiment, a composition is provided comprising an scFv that binds an determinant on the surface of a red blood cell, bound to thrombomodulin or a domain thereof. In a further embodiment, the determinant is a human glycophorin A. In a further composition, the determinant is a non-primate analogue of human glycophorin e.g., murine glycophorin A associated protein. In another embodiment, the determinant is an ABO blood group antigen. In another embodiment, the determinant is RBC band 3 antigen. In another embodiment, the determinant is expressed on the surface of a red blood cell at a density greater than 5,000 copies per red blood cell. In one embodiment, a composition comprises the extracellular domain of thrombomodulin, bound to a scFv that binds glycophorin A. In another embodiment, a composition comprises a scFv that binds glycophorin A, bound to the extracellular domain of thrombomodulin. In a further embodiment, a composition comprises an scFv that binds glycophorin A, bound to the lectin-like domain of thrombomodulin. In still a further embodiment, a composition comprises a scFv that binds glycophorin A, bound to an EGF-like domain of thrombomodulin. In other embodiments, the EGF-like domain is EGF3-6, EGF4-6, or EGF5-6.

In one embodiment, compositions targeted to treating, inhibiting, preventing, or alleviating inflammation or a disorder associated with inflammation, comprise an scFv that binds determinant on the surface of an endothelial cell or on the surface of a red blood cell, bound to the lectin-like domain of thrombomodulin. In further embodiments, the scFv of the composition binds PECAM-1, ICAM-1, or VCAM-1. In still other embodiments, the scFv of the composition binds glycophorin A (or a glycophorin A associated protein), an ABO blood group antigen, or RBC band 3 antigen.

In another embodiment, compositions targeted to treating, inhibiting, preventing, or alleviating coagulation or a disorder associated with coagulation (or thrombosis), comprise an scFv that binds a determinant on the surface of an endothelial cell or on the surface of a red blood cell, bound to an EGF-like domain of thrombomodulin. In further embodiments, the EGF-like domain is EGF3-6, EGF4-6, or EGF5-6. In still a further embodiment, the EGF-like domain is EGF4-6. In still further embodiments, the scFv of the composition binds PECAM-1, ICAM-1, or VCAM-1. In still other embodiments, the scFv of the composition binds glycophorin A (or a glycophorin A associated protein), an ABO blood group antigen, or RBC band 3 antigen.

### V. PHARMACEUTICAL COMPOSITIONS AND METHODS OF ADMINISTRATION

Pharmaceutical compositions containing a composition described herein and a pharmaceutically acceptable carrier or vehicle as described herein are useful for the treatment of a variety of diseases and disorders. In one embodiment, a composition comprises a pharmaceutically acceptable vehicle for intravenous administration. In another embodiment, a composition comprises a pharmaceutically acceptable vehicle for administration via other vascular routes, including but not limited to, intra-arterial and intra-ventricular administration, as well as routes providing slower delivery of drugs to the bloodstream such as intramuscular, intra-peritoneal or intra-cutaneous administration to an animal in need thereof. As used herein, the terms “animal” and “patient” include any mammal. In a further embodiment, the terms “animal” and “patient” refer to a human.

Pharmaceutically acceptable vehicles/carriers include any of those conventionally used in the art, e.g., saline, phosphate buffered saline (PBS), or other liquid sterile vehicles accepted for intravenous injections in clinical practice. Pharmaceutical compositions may also include buffers, pH adjusting agents, and other additives conventionally used in medicine. In one embodiment, compositions described herein are administered systemically as a bolus intravenous injection of a single therapeutic dose of the composition. In a further embodiment, the dose is 0.1-1.0 mg/kg. In another embodiment, the dose is 0.05-0.5 mg/kg.

In one embodiment, a method of delivering a thrombomodulin domain to a luminal surface of vascular endothelium is provided comprising delivering a composition protein as described herein, or a pharmaceutical composition described herein, to a blood vessel. In another embodiment, a method of delivering a thrombomodulin domain to the surface of a red blood cell is provided comprising delivering a composition as described herein, or a pharmaceutical composition described herein, to a blood vessel. In one embodiment, compositions described herein are administered via a systemic intravascular route, e.g., a vascular catheter. In some embodiments, rapid targeting of an organ or system may be accomplished by delivery via coronary artery (e.g., for prophylaxis of acute myocardial infarction (AMI)) or the cerebral artery (e.g., for prophylaxis of stroke and other cerebrovascular thrombotic events). Further, compositions described herein may be administered prophylactically, i.e., in patients predisposed to thrombosis. In a further embodiment, compositions described herein may be administered to an organ donor, utilized with an isolated organ transplant (e.g., via perfusion), or used with vascular stents.

Thus, methods of treating, inhibiting or preventing a cardiovascular disorder, such as thrombosis, tissue ischemia, acute myocardial infarction (AMI), non-segmented elevated AMI, deep vein thrombosis, ischemic stroke, hyperoxic injury, transient ischemic attack (TIA), cerebrovascular disease, disseminated intravascular coagulation (DIC), pulmonary embolism, or ischemic peripheral vascular disease, involves administering a composition as described herein, or a pharmaceutical composition as described herein, to a blood vessel in a mammal in need thereof In such disorders, the thrombomodulin domain and its dosage in delivery may be selected and adjusted by an attending physician with regard to the nature of the disorder, the physical condition of the patient, and other such factors. The selection of the cleavage site in a composition may also be selected to match the disorder, e.g., a thrombin cleavage site for treating or preventing thrombosis, if thrombotic pathways predominate in the pathogenesis of a given type of disorder in a given patient.

Similarly, in other embodiments, methods of treating, inhibiting or preventing inflammation, pulmonary edema, sepsis, acute lung injury (ALI), acute respiratory distress syndrome (ARDS), aseptic systemic inflammation, and other inflammatory conditions are provided by administering the appropriately designed composition, according to the teachings of this specification, if inflammatory pathways predominate in the pathogenesis of a given type of disorder in a given patient.

Further, in other embodiments, these compositions can be used to treat both thrombotic and inflammatory components of all diseases conditions listed in two previous paragraphs, because these conditions are known to intertwine these two pathological pathways, as well as other mechanisms of tissue injury including apoptosis, because TM has anti-apoptotic effects, if combined thrombotic, inflammatory and/or apoptotic pathways predominate in a given disorder in a given patient.

Also provided is the use of a composition or a pharmaceutical composition as described herein as a medicament. Further provided is the use of a composition or a pharmaceutical composition as described herein to treat any of the above conditions.

### VI. EXAMPLES

The examples that follow do not limit the scope of the embodiments described herein. One skilled in the art will appreciate that modifications can be made in the following examples which are intended to be encompassed by the spirit and scope of the invention.

### Example 1

### Preparation of an Anti-PECAM-1 scFv/Thrombomodulin (TM) Extracellular Domain Composition and Soluble TM (sTM)

Total RNA was extracted from mouse lung and reverse transcribed to cDNA. Mouse thrombomodulin (TM) extracellular domain (Leu¹⁷-Ser⁵¹⁷) was amplified by PCR using primers fmTMsen: 5′-ATAAGAATGCGGCCGCACTCTCCGCACTA GCC-3′ (SEQ ID NO: 7) and fmTMrev1: 5′-GTCATGGTCTTTGTAGTCAGAGTG CACTGGCCTTG-3′ (SEQ ID NO: 8). The product was amplified again with fmTMsen and fmTMrev2: 5′-GCTCGAGTCATCACTTGTCATCGTCAT CCTTGTAATCGATATCATGATCTTTATAATCACCGTCATGGTCTTTG TAGTC-3′ (SEQ ID NO: 9), which appends a triple-FLAG affinity peptide tag at 3′ end.

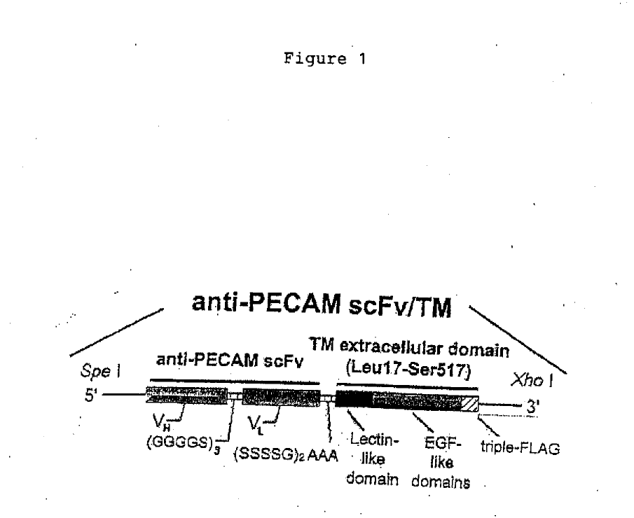

The resultant fragment was subcloned into the construct described in Ding, et al. [Endothelial targeting of a recombinant construct of a PECAM-1 single-chain variable antibody fragment (scFv) with prourokinase facilitates prophylactic thrombolysis in the pulmonary vasculature, Blood 2005; 106:4191-4198], generating the scFv/TM construct (FIG. 1). A soluble thrombomodulin sTM construct was similarly produced. Generation of drosophila cells expressing scFv/TM or sTM was performed as described previously. Id. Proteins were purified by anti-FLAG affinity chromatography and analyzed on SDS-PAGE gels after incubation with or without 50 mM dithiothreitol (DTT). scFv and sTM migrated at 48 and 56 kDa, respectively, under non-reduced conditions, and exhibited the expected slight upward shift after reduction, confirming the disruption of the compact secondary structure dependent on disulfide bonding.

### Example 2

### Protein C Activation and PECAM-1 Binding of anti-PECAM-1 scFv/TM Extracellular Domain Composition vs. sTM

Protein C Activation

anti-PECAM-1 scFv/TM and sTM (10 nM) was incubated with thrombin (10 nM; bovine thrombin obtained from Amersham Biosciences (Piscataway, N.J.)) and protein C (100 nM or 300 nM; American Diagnostica, Inc. (Stamford, Conn.)) in Tris buffer containing 30 mM imidazole, 0.2 mM NaCL, 1 mM CaCl₂(pH 8.0). After one hour incubation, hyrudin (40 U/ml; Sigma (St Louis, Mo.)) was added to terminate thrombin activity. Activated protein C (APC) amidolytic activity was measured by optical density using Spectrozyme® PCa chromogenic substrate (American Diagnostica, Inc. (Stamford, Conn.)).

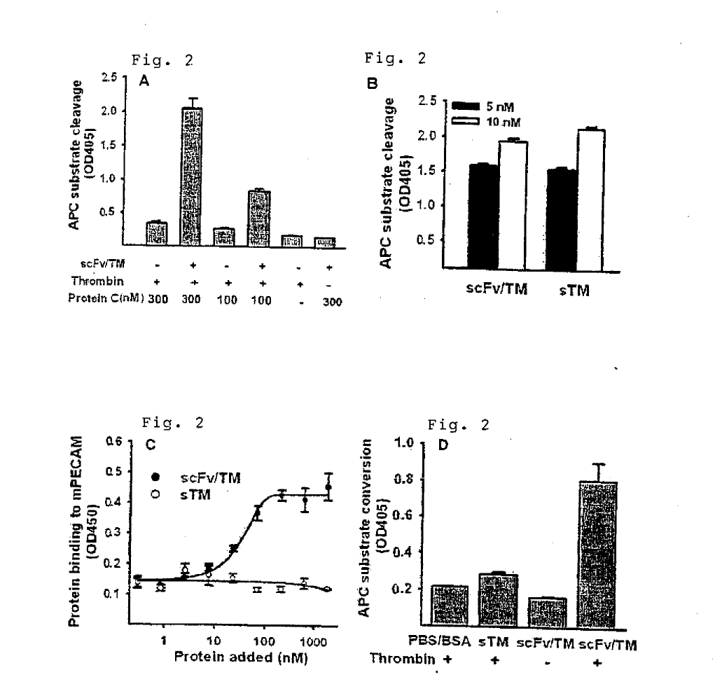

scFv/TM induced protein C activation (to activated protein C, APC) in a thrombin-dependent manner (FIG. 2A). Further, scFv and sTM induced protein C activation to the same extent at 5 nM and 10 nM concentrations (FIG. 2B).

PECAM-1 Binding

Binding of scFv/TM to mouse PECAM-1 was measured by ELISA using antibody against mouse TM (2 μg/ml), as described in Ding, et al. [Prophylactic thrombolysis by thrombin-activated latent prourokinase targeted to PECAM-1 in the pulmonary vasculature, Blood 2008; 111:1999-2006]. Wells were coated with 25 μg/ml mouse PECAM-1 and pre-incubated with either anti-PECAM scFv/TM or sTM. Protein C was added with or without thrombin (FIG. 2D), and activated protein C activity was measured as described above.

scFv/TM, but not sTM, bound to mouse PECAM-1 (FIG. 2C). In the presence of thrombin, PECAM-bound scFv/TM produced activated protein C (APC), whereas significantly less APC was detected in wells pre-incubated with sTM (FIG. 2D).

### Example 3

### Organ Distribution of anti-PECAM-1 scFv/TM Extracellular Domain Composition vs. sTM after Intravenous Injection

Organ Distribution

Male C57BL/B6 mice, 6-10 weeks of age, were used in experiments performed in accordance with NIH guidelines and approved by the University of Pennsylvania IACUC. Anesthetized mice were injected intravenously with 50 μg of scFv/TM or equimolar amounts of sTM and sacrificed 1 hour later to obtain organ homogenates as previously described in Ding, et al. [Prophylactic thrombolysis by thrombin-activated latent prourokinase targeted to PECAM-1 in the pulmonary vasculature, Blood 2008; 111:1999-2006].

Anti-FLAG immunoblot was used to detect triple-FLAG tagged scFv/TM and sTM in the tissue homogenates of mice injected with these proteins. (Anti-FLAG M2 affinity gel and mouse monoclonal antibody were from Sigma (St Louis, Mo.)) To assess the amounts of scFv/TM in lung homogenates, the purified protein was serially diluted and blotted in adjacent lanes to the actin (Santa Cruz Biotechnology, Inc (Santa Cruz, Calif.)) standard curve.

Following intravenous injection, scFv/TM, but not sTM, accumulated in mouse lungs. Further, ˜35% of the injected dose of scFv/TM accumulated per gram of lung. scFv accumulated preferentially in the pulmonary vasculature (lung) relative to plasma, heart, spleen kidney and liver.

Localization of scFv in Lung

Mice were injected with 200 μg of scFv/TM or the same amount of sTM. The perfused lungs were used to make 5 μm cryostat sections. Acetone-fixed sections were incubated sequentially with goat anti-VE-Cadherin antibody (Santa Cruz Biotechnology, Inc (Santa Cruz, Calif.)) or anti-FLAG antibody (Sigma, St Louis, Mo.) in PBS containing 1% BSA and 10% calf serum, FITC-labeled anti-goat IgG (Jackson ImmunoResearch Laboratories, West Grove, Pa.) and M.O.M Immunodetection Kit (Vector, Burlingame, Calif.). No appreciable staining was observed in isotype controls. Immunostaining showed that scFv/TM, but not sTM, bound to the pulmonary endothelium following IV injection in mice.

### Example 4

### Binding of the scFv//TM to Ter119

A Ter119scFv-TM (Ter119 is a mouse glycophorin A-associated protein) composition was prepared as previously described.

Agglutination (Aggregation) Assay

Mouse and human red blood cells were loaded with the composition, and the cells were incubated with an equimolar concentration of cytokine HMGB1 for 30 minutes at room temperature. Following incubation, a polyclonal antibody specific for HMGB1 was added. The preparations were incubated with the polyclonal antibody in V-shaped plates for 20 minutes at 25° C. and centrifuged at 1200 g for 2 minutes to precipitate RBCs. Optical density was measured.

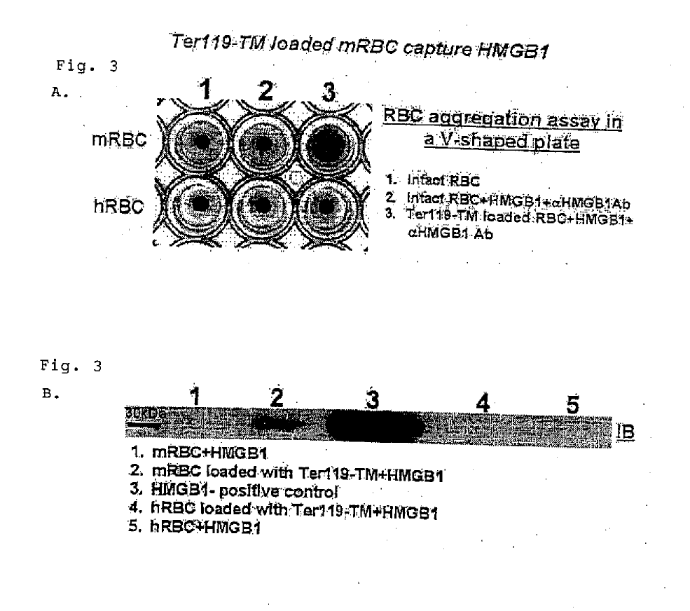

Only Ter119scFv-TM loaded RBCs incubated with HMGB1 and then polyclonal anti-HMGB 1 antibody aggregated, as reflected in FIG. 3A. This result confirms that the thrombomodulin domain of the scFv-TM composition binds the pro-inflammatory mediator HMGB 1.

Western Blot confirmed Ter119scFv-TM's binding to HMGB1, as well as Ter119′s specificity for mouse red blood cells. See FIG. 3B.

### Example 5

### Activity of Expressed scFv/TM Composition

A Ter119scFv/TM was prepared as previously described. Mouse and human red blood cells (mRBC and hRBC, suspended to 10% hematocrit) were loaded with Ter119scFv/TM by incubation for one hour at 37° C. Unbound ligand was removed via centrifugation with PBS-BSA (phosphate buffered saline-bovine serum albumin). Loaded and intact (non-loaded) RBC were incubated with thrombin and protein C. Activation of protein C was measured by spectrazyme assay at λ450.

Only mRBC incubated with Ter119/scFvTM caused activation of protein C in the presence of thrombin, as reflected in FIG. 4. This result confirms the binding specificity of Ter119/scFvTM and the functional activity of Ter119/scFvTM when bound to the red blood cell.

### Example 6

### Biodistribution of scFv/TM in vivo

A. ⁵¹Cr labeled RBCs loaded with Ter119scFv/TM and RBCs loaded with Ter119scFv/TM-¹²⁵I were prepared and injected intravenously in wild type (WT) mice. (TM was radiolabeled with ¹²⁵I-Na (Perkin Elmer, Wellesley, Mass.) using Iodogen (Pierce, Rockford, Ill.)). At one hour, three hours, and six hours of circulation, tissue uptake was determined. RBC-bound radioactivity was measured in a γ-counter (Perkin Elmer).

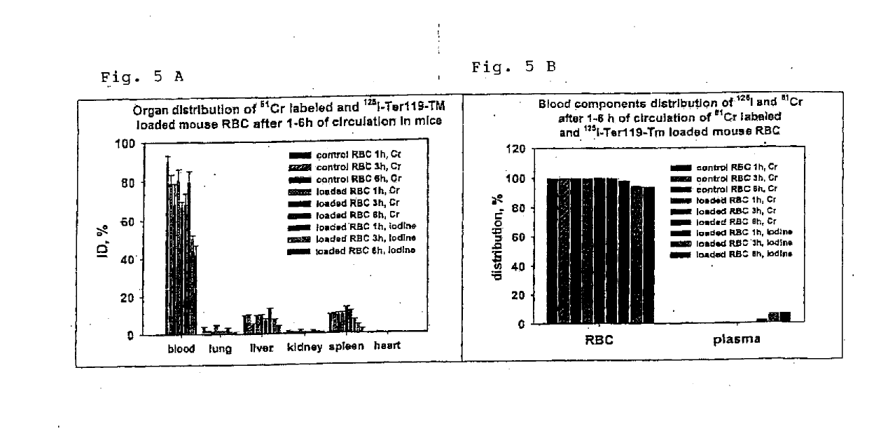

FIG. 5A shows a lack of accumulation of loaded RBCs in the lungs, reflecting the absence of aggregation, and lack of accumulation in the spleen reflects the absence of damage to the RBCs. FIG. 5B reflects no significant detachment of composition from RBCs within 6 hours.

B. Long-Term Biodistribution of scFv-TM in viva

RBCs loaded with Ter119scFv/TM-¹²⁵I were injected intravenously in wild type (WT) mice. At one-half hour, one hour, three hours, six hours, and twenty-four hours of circulation, tissue uptake was determined. RBC-bound radioactivity was measured in a γ-counter (Perkin Elmer).

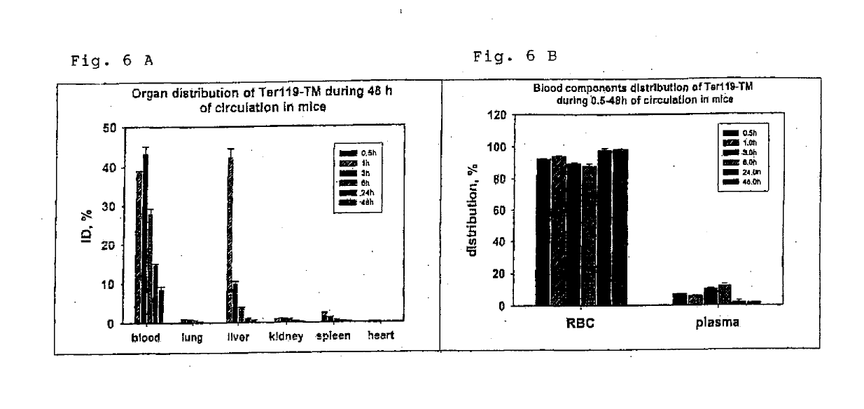

FIG. 6A shows a lack of accumulation of loaded RBCs in the lungs, reflecting the absence of aggregation, and lack of accumulation in the spleen reflects the absence of damage to the RBCs. FIG. 6B reflects no significant detachment of composition from RBCs within 6 hours.

C. Stable and Prolonged Loading via Intraperitoneal Delivery

RBCs loaded with Ter119scFv/TM-¹²⁵I were injected intraperitoneal (IP) in wild type (WT) mice. At one hour and three hours of circulation, tissue uptake was determined. RBC-bound radioactivity was measured in a γ-counter (Perkin Elmer).

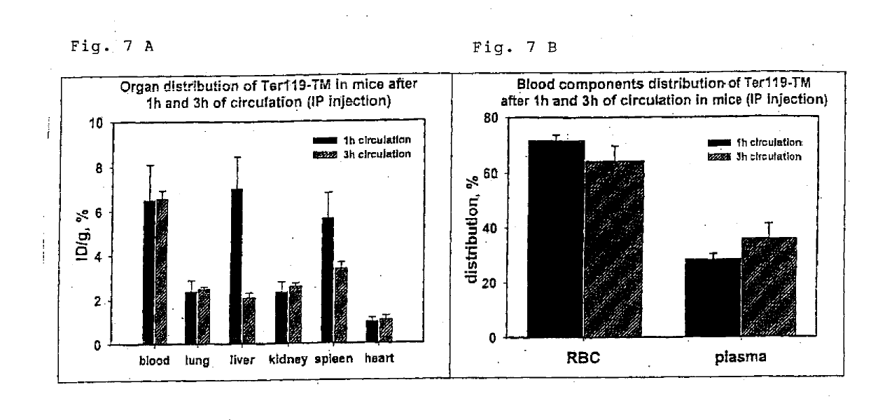

FIGS. 7A and 7B reflect that intraperitoneal (IP) delivery offers stable and prolonged loading of RBCs. IP delivery provides for chronic and repetitive use.

### Example 7

### Protective Effect of anti-PECAM-1 scFv/TM Extracellular Domain Composition vs. sTM Administered Prior to Lung Ischemia/Reperfusion Injury

Left lung ischemia and reperfusion, tissue harvest, and protein extraction were performed as described in Ding, et al. [Prophylactic thrombolysis by thrombin-activated latent prourokinase targeted to PECAM-1 in the pulmonary vasculature, Blood 2008; 111:1999-2006]. Fibrin β-chain monoclonal antibody was obtained from American Diagnostica, Inc. (Stamford, Conn.). Thirty minutes prior to ischemia/reperfusion (I/R) injury, mice were injected with 50 μg scFv/TM, an equimolar amount of sTM, or PBS. Following ischemia (120 minutes) and reperfusion (150 minutes), TM and fibrin bands were detected by immunoblotting, optical density (OD) measured and normalized to the actin band. Change in protein level was calculated as fold or percent of a protein in injured lung relative to sham-operated lung.

Anticoagulant Effect of scFv/TM

Following I/R injury in untreated mice, fibrin deposition in lung increased and TM levels decreased. However, when scFv/TM and sTM were introduced 30 minutes prior to I/R, scFv/TM decreased the fibrin deposition in the lung by ˜70% (but not sTM).

Antiinflammatory Effect of scFv/TM

In addition, following I/R injury, nuclear extracts were prepared using a nuclear isolation kit (Pierce, Rockford, Ill.) and subjected to anti-Egr-1 (Santa Cruz Biotechnology, Inc (Santa Cruz, Calif.)) immunoblotting. Egr(early growth response)-1 is a key transcription factor involved in the inflammatory response to I/R. Egr-1 was quantified by OD and expressed as a ratio relative to the stable factor SP1 (Santa Cruz Biotechnology, Inc (Santa Cruz, Calif.)) in the same extracts. Lung myeloperoxidase (MPO) activity was measured using Myeloperoxidase Assay Kit from CytoStore (Alberta, Canada). Oxygen tension in the arterial blood was measured, as described in Ding, et al. [Prophylactic thrombolysis by thrombin-activated latent prourokinase targeted to PECAM-1 in the pulmonary vasculature, Blood 2008; 111:1999-2006].

Consistent with the literature, I/R caused elevation of the pulmonary level of Egr-1 (˜8× sham). scFv/TM suppressed Egr-1 elevation (˜3× sham, vs. ˜6× sham for sTM), as well as the leukocyte marker LPO (˜3.5× sham). For LPO, scFv/TM was ˜2× sham vs. ˜3× sham for sTM. Further, scFv/TM preserved the arterial oxygen pressure (˜450 mmHg) after I/R more effectively than sTM (˜350 mmHg), vs. ˜250 mmHg with PBS control.

### Example 8

### Anti-PECAM-1 scFv/TM Extracellular Domain vs. sTM in Acute Inflammatory Lung Injury (ALI)

LPS/hyperoxia Suppression of TM and Induction of ALI

Acute lung injury was induced in anesthetized mice by intratracheal injection of endotoxin (Sigma, Lipopolysaccharides (LPS) from E. coli, 0127:B8, 8 mg/kg). Once the incision was closed with a wound clip, mice were exposed to 98% O₂ (hyperoxia) for 18 hours and then sacrificed. Sham-operated mice were injected with PBS and exposed to room air. Lung proteins were extracted and levels of TM, high-mobility group-B 1 (HMGB1), and fibrin deposition were analyzed as described in the preceding examples.

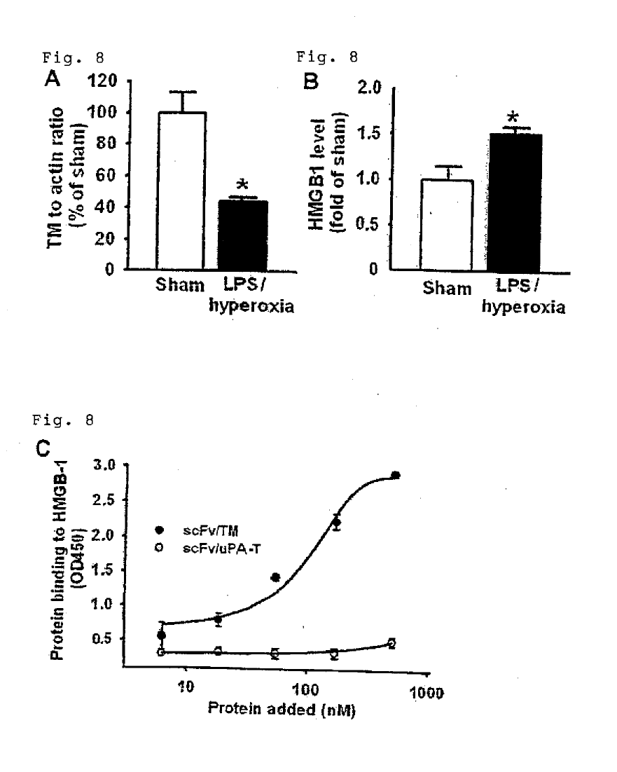

LPS/hyperoxia down regulated endogenous TM in the lungs (FIG. 8A) to the extent seen in the I/R model (˜60% and 50%, respectively), although only modest fibrin deposition in the lungs was observed. In contrast, pulmonary level of HMGB1, an inflammatory cytokine, was elevated in LPS/hyperoxia (FIG. 8B), but not in I/R.

Binding of anti-PECAM-1 scFv/TM Extracellular Domain Composition to HMGB1

Thrombomodulin has been reported to bind and neutralize HMGB1. Binding of anti-PECAM scFv/TM to HMGB1 was measured by ELISA. A construct containing anti-PECAM scFv fused with thrombin-activatable uPA (scFv/uPA-T; See Ding, et al., Blood 2008, cited above) tagged with triple FLAG (Sigma, St. Louis, Mo.) as a control. Wells coated with HMGB1 (5 μg/ml) were incubated with compositions, and the binding was detected using anti-FLAG antibody, HRP (horseradish peroxidase)-conjugated anti-mouse IgG and TMB (3.3′,5.5′-tetramethylbenzidine) substrate (OD_{450 nm}; Pierce, Rockford, Ill.). Immunoprecipitation-Western Blot (IP-WB) was performed to compare the HMGB1 binding of scFv/TM and sTM. HMGB1 (10 μg/ml) was incubated with scFv/TM (8 μg/ml), the same amounts of sTM or scFv/uPA-T, followed by addition of anti-FLAG agarose (Sigma). Agarose beads were solubilized and subjected to anti-HMGB1 Western blot (Sigma).

FIG. 8C reflects that scFv binds HMGB 1 in a dose-dependent manner. Immunoprecipitation-Western blot (IP-WB) confirmed comparable binding of HMGB1 to scFv/TM and sTM (vs. control (scFv/uPA-T)), suggesting that scFv/TM has antiinflammatory effect.

Antiinflammatory Effects of anti-PECAM-I scFv/TM Extracellular Domain Composition in Acute Lung Injury (ALI)

Anesthetized mice were injected intravenously at time points before or after onset of injury. In the pre-injury injection group (‘pre-inject’), 50 μg scFv/TM and equimolar amounts of sTM or PBS (control) were injected intravenously into mice 30 minutes prior to injury. In the post-injury injection group (‘post-inject’), 50 μg scFv/TM and equimolar amounts of sTM or PBS (control) were injected intravenously one hour after injury.

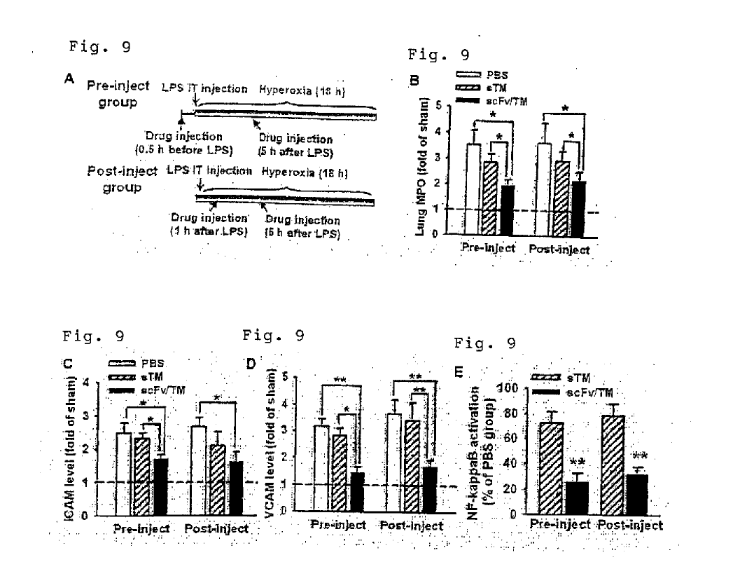

Acute lung injury was induced by intratracheal injection of endotoxin (Sigma, Lipopolysaccharides (LPS) from E. coli, 0127:B8, 8 mg/kg). Once the incision was closed with a wound clip, mice were exposed to 98% O₂ (hyperoxia) for 18 hours and then sacrificed. In both groups, 50 μg scFv/TM and equimolar amounts of sTM or PBS (control) were also injected five hours after injury (LPS administration). The experiment is summarized in FIG. 9A. After sacrifice, lung total proteins were extracted. ICAM-1 and VCAM-1 were detected by immunoblotting, quantified using densitometry, and normalized to actin, as described in the preceding examples. Lung MPO activity and nuclear extraction were performed as described above. To detect activation of NF-κβ, 10 μg nuclear extracts were mixed with biotinylated NF-κβ-binding probe (Panomics, Fremont, Calif.) and analyzed using an electrophoretic mobility shift assay (EMSA) kit (Panomics). Attenuation of NF-κβ activation was assessed by comparing the OD of shifted bands among groups.

scFv/TM, but not sTM, injected either before and after the insult, blunted the increase in MPO (FIG. 9B) and expression of pro-inflammatory cell adhesion molecules ICAM-1 and VCAM-1 seen in the lungs of LPS/hyperoxia challenged mice (FIGS. 9C, 9D). Further, scFv/TM (but not sTM) injected before or after insult inhibited NFκB activation by ˜70% compared with unprotected animals (FIG. 9E), thereby blocking one of the primary inflammatory pathways involved in ALI and sepsis. Pre-injection of even higher amounts of scFv/uPA-T induced little, if any additional suppression of pulmonary NF-κB and MPO in this model, indicating that it is unlikely that these anti-inflammatory effects of scFv/TM are due to inhibition of thrombosis or blockage of PECAM-1.

### Example 9

### Anti-PECAM-1 scFv/TM Extracellular Domain vs. sTM in Amelioration of Pulmonary Edema

Increased vascular permeability and edema are pathological hallmarks of acute lung injury (ALI). To test the effects of scFv/TM, scFv/TM, sTM or PBS were injected after lung injury (‘post-inject’ group of Example 5, FIG. 4A), and lung wet/dry ratio was measured, as described in Kozower B D, et al. [Immunotargeting of catalase to the pulmonary endothelium alleviates oxidative stress and reduces acute lung transplantation injury. Nat Biotechnol. 2003; 21:392-398].

To measure vascular permeability, 150 μl of Evans blue (5 mg/ml; Sigma) was injected intravenously one hour before sacrifice. The lungs were perfused and homogenized with formamide. Evans blue was extracted from homogenates by incubation at 55° C. for 24 hours followed by centrifugation. OD_{620 nm} was measured in the supernatants and quantified using a standard curve of the dye optical density.

To measure bleeding time, mice were injected intravenously with 50 μg scFv/TM, 20 μg activated protein C (APC), or PBS. Bleeding time was analyzed 60 minutes after injection by the tail clip method, described in Cheng Y, et al. [Cyclooxygenases, microsomal prostaglandin E synthase-1, and cardiovascular function. The Journal of Clinical Investigation. 2006; 116:1391-1399.]

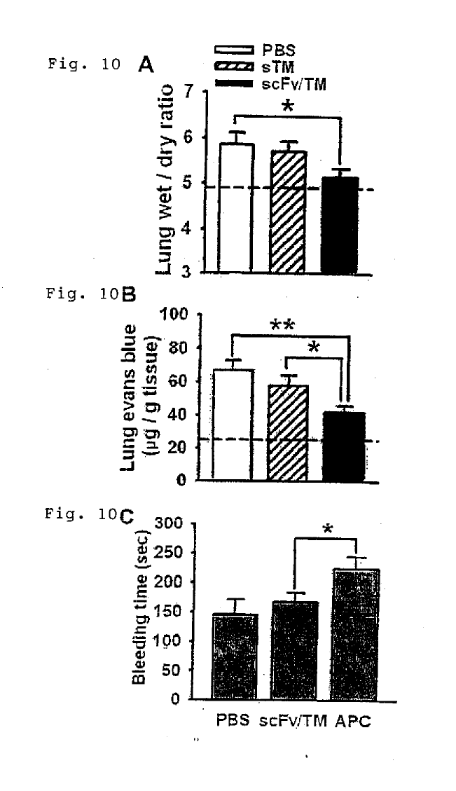

scFv/TM, but not sTM, injected after initiation of LPS/hyperoxia injury decreased pulmonary edema, assessed by both lung wet/dry ratio (FIG. 10A) and Evans blue extravasation (FIG. 10B). Bleeding times were not prolonged in mice injected with scFv/TM at an effective dose used in I/R and LPS/hyperoxia models. In contrast, injection of APC at a dose that blunted pulmonary edema comparably with scFv/TM significantly prolonged bleeding times (FIG. 10C).

### Example 10

### Preparation of scFv/Thrombomodulin (TM) Constructs

Constructs (compositions) are prepared according to Example 1, using PCR amplification of the relevant thrombomodulin domain. scFvs are prepared as described therein or as follows.

Preparation of scFv

scFvs are generated in accordance with the teachings herein, as well as those of Spitzer, et al. (Mol. Immunol. 2003, 40:911-919). Total RNA of a hybridoma cell line is isolated (e.g., by RNeasy, Qiagen, Velencia, Calif.). RNA is reverse transcribed, e.g., using the SMART™ technology (Clontech, Palo Alto, Calif.) employing known primers (e.g., those of Dübel, et al. (J. Immunol. Methods 1994, 175:89-95)). The resulting heavy (VH) and light (VL) chain variable cDNA fragments are subcloned into a suitable plasmid, e.g., the pCR®2.1-TOPO® (Invitrogen, Carlsbad, Calif.). Plasmids are then transfected into E. coli. VH and VL chains are then isolated by conventional techniques, e.g., agarose gel column or gel electrophoresis.

V_{H} and V_{L} chains are combined with a suitable linker, e.g., a (G₄S)₃ linker (Böldicke, et al. (Stem Cells 2001, 19:24-36)) resulting in the desired scFv. PCR-derived sequences are verified by DNA sequencing. The amino acid primary sequence may be analyzed to determine complementarity determining regions (CDRs) by application of the rules described at: Antibody Structure and Sequence Information V2.0 (http://www.rubic.rdg.ac.uk).

Compositions

The following compositions are prepared:
anti-ICAM scFv/thrombomodulin extracellular domain;
anti-VCAM scFv/thrombomodulin extracellular domain;
anti-glycophorin A (GPA) scFv/thrombomodulin extracellular domain;
anti-PECAM scFv/thrombomodulin lectin-like domain;
anti-ICAM scFv/thrombomodulin lectin-like domain;
anti-VCAM scFv/thrombomodulin lectin-like domain;
anti-glycophorin A (GPA) scFv/thrombomodulin lectin-like domain;
anti-PECAM scFv/thrombomodulin EGF-like domain;
anti-ICAM scFv/thrombomodulin EGF-like domain;
anti-VCAM scFv/thrombomodulin EGF-like domain;
anti-glycophorin A (GPA) seFv/thrombomodulin EGF-like domain;
anti-PECAM scFv/thrombomodulin EGF4-6-like domain;
anti-ICAM scFv/thrombomodulin EGF4-6-like domain;
anti-VCAM seFv/thrombomodulin EGF4-6-like domain; and
anti-glycophorin A (GPA) scFv/thrombomodulin EGF4-6-like domain.

In mouse thrombomodulin, EGF1-6 extends from amino acid 240 (Gly) through aa 480 (Phe) of SEQ ID NO: 3. EGF 4-6 extends from amino acid 364 (Leu) through aa 480 (Phe) of SEQ ID NO: 3. The N-terminal lectin-like domain extends from aa 17 (Leu) through aa 243 (Asn) of SEQ ID NO: 3. In mouse models, the anti-glycophorin A compositions are prepared using anti-mouse Ter119 in place of anti-GPA.

### Example 11

### Protective effect of scFv/TM Compositions vs. sTM Administered Prior to Lung Ischemia/Reperfusion Injury

Compositions prepared in Example 10 are tested according to Example 7. Following I/R injury in untreated mice, fibrin deposition in lung is increased and TM levels are decreased. However, when scFv/TM and sTM are introduced 30 minutes prior to I/R, scFv/TM decreases the fibrin deposition in the lung (but not sTM).

I/R causes elevation of the pulmonary level of Egr-1 (˜8× sham). scFv/TM suppresses Egr-1 elevation as well as the leukocyte marker LPO. Further, scFv/TM preserves the arterial oxygen pressure after I/R more effectively than sTM.

### Example 12

### scFv/TM Compositions vs. sTM in Acute Inflammatory Lung Injury (ALI)

Compositions prepared in Example 10 are tested according to Example 8. scFv binds HMGB1 in a dose-dependent manner. Immunoprecipitation-Western blot (IP-WB) confirms comparable binding of HMGB1 to scFv/TM and sTM (vs. control (scFv/uPA-T)).

scFv/TM, but not sTM, injected either before and after the insult, blunts the increase in MPO (FIG. 7B) and expression of pro-inflammatory cell adhesion molecules ICAM-1 and VCAM-1 seen in the lungs of LPS/hyperoxia challenged mice. Further, scFv/TM (but not sTM) injected before or after insult inhibits NFκB activation by ˜70% compared with unprotected animals, thereby blocking one of the primary inflammatory pathways involved in ALI and sepsis. Pre-injection of even higher amounts of scFv/uPA-T induces little, if any additional suppression of pulmonary NF-κB and MPO in this model, indicating that it is unlikely that these anti-inflammatory effects of scFv/TM are due to inhibition of thrombosis or blockage of the targeting domain.

### Example 13

### scFv/TM Compositions vs. sTM in Amelioration of Pulmonary Edema

Compositions prepared in Example 10 are tested according to Example 9. scFv/TM, but not sTM, injected after initiation of LPS/hyperoxia injury decreases pulmonary edema. Bleeding times are not prolonged in mice injected with scFv/TM at an effective dose used in I/R and LPS/hyperoxia models. In contrast, injection of APC at a dose that blunted pulmonary edema comparably with scFv/TM significantly prolongs bleeding times.

### Example 14

### In vivo Thrombolysis in a Model of Intravascular Thrombolysis in Mice—scFv/TM

The method of dissolution of carotid arterial thrombi according to J. Murciano, et al., Nature Biotechnology, 21(8): 891-896, 895 (August 2003) was used. A Ter119scFv/TM composition (prepared according to Example 7), PBS, and sTM (equimolar) are administered via injection via jugular vein. 30 minutes after administration, acute vascular trauma is induced by administration of 15% FeCl₃ (2 minutes flow).

Injection of non-targeted sTM does not affect rapid thrombotic occlusion of the artery compared with mice pre-treated with placebo (PBS). sTM does not attenuate nor delay artery occlusion (defined as complete cessation of blood perfusion by Doppler ultrasound). However, the equimolar dose of scFv-TM composition significantly delays occlusion time.

The data indicates that the scFv/TM thromboprophylaxis in animal models.

### Example 15

### Prophylactic Thrombolysis of Cerebrovascular Thrombi

The protocol of K. Danielyan, et al., J. Pharm. and Exp. Therapeutics, 321(3): 947-952, 948 (June 2007) is used.

A Ter119scFv/TM composition prepared according to Example 7, sTM, and PBS (equimolar), are administered in a standard 120-μL volume of PBS via catheter inserted into the right femoral vein of anesthetized mice. 30 minutes after administration, a suspension of ¹²⁵I-fibrin emboli is injected via the right middle cerebral artery. 30 minutes later, mice are sacrificed and ¹²⁵I content of the brain is measured to determine the extent of cerebrovascular thrombolysis based on the amount of residually radiolabeled clots residing in the brain.

Residual activity for the scFv/TM composition mice is nearly three times lower than that for PBS mice alone. These data indicate that the scFv/TM compositions provide thromboprophylaxis of cerebrovascular thrombi in animal models.

### Example 16

### Binding of scFv/TM Composition to Glycophorin A

A. Generation of human anti-glycophorin A (hGPA) scFv/TM

The scFv with specificity for hGPA is generated essentially as described above (Spitzer, et al., Mol. Immunol., 40:911-919 (2004)) from the mouse hybridoma cell line BRIC 256 (Anstee, et al, Eur. J. Immunol., 12:228-232 (1982). This line secretes an IhG1 mAb that recognizes a blood group-independent epitope on human GPA (Gardner, et al, Immunology, 68:283-289 (1989)). Total RNA is isolated (RNeasy™; Qiagen). Reverse transcription, followed by PCR (RT-PCR), is conducted using the SMART™ technology (BD Clontech) using primer combinations described previously (Dubel, et al., J. Immunol. Methods, 175:89-95 (1994). The resulting H and L chain variable cDNA fragments are subcloned into pCR2.1-TOPO (Invitrogen Life Technologies). After introducing suitable flanking restriction sites via PCR, the V_{H} and Vₜ chains are combined with a (G₄S)₃ linker resulting in the scFv Bric-256. Analysis of the amino acid primary sequence to determine the complementarity determining regions of the scFv Bric-256 is performed by applying the rules described at Ab Structure and Sequence Information version 2.0 (www.rubic.rdg.ac.uk).

B. An hGPAscFv/TM is prepared as described herein for other scFv/TMs. sTM is radiolabeled with ¹²⁵I-Na (Perkin Elmer, Wellesley, Mass.) using Iodogen (Pierce, Rockford, Ill.). Mouse and human red blood cells (mRBC and hRBC, suspended to 10% hematocrit) are loaded at the same concentration with hGPA-scFv/TM by incubation for one hour at 37° C. Unbound ligand is removed via centrifugation with PBS-BSA (phosphate buffered saline-bovine serum albumin) and RBC-bound radioactivity is measured in a γ-counter (Perkin Elmer).

Specific binding of anti-GPA scFv/TM to hRBC is revealed vs. mRBC.

### Sequence Listing Free Text

The following information is provided for sequences containing free text under numeric identifier <223>.

| SEQ ID NO: | |
|---|---|
| (containing free text) | Free text under <223> |
| 4 | linker based on Homo sapiens |
| 5 | linker based on Homo sapiens |
| 6 | cleavage site based on Homo sapiens |
| 7 | primer based on Mus Musculus |
| 8 | primer based on Mus Musculus |
| 9 | primer based on Mus Musculus |

All documents referenced herein, U.S. Provisional Patent Application No. 61/105,956, filed Oct. 16, 2008, and the Sequence Listing filed herewith, are incorporated by reference. It will be clear to one of skill in the art that modifications can be made to the specific embodiments described herein without departing from the scope of the invention.

1. A composition comprising a thrombomodulin domain linked to a targeting moiety that binds to a determinant on the surface of a target cell.

2. The composition according to claim 1, wherein the thrombomodulin domain is the extracellular domain of thrombomodulin.

3. The composition according to claim 1, wherein the thrombomodulin domain is the N-terminal lectin-like domain of thrombomodulin.

4. The composition according to claim 1, wherein the thrombomodulin domain is an epidermal growth factor (EGF)-like domain of thrombomodulin.

5. The composition according to claim 1, wherein the targeting moiety is a single chain antigen-binding domain (scFv).

6. The composition according to claim 1, wherein the targeting moiety and thrombomodulin domain are linked as a continuous polypeptide chain.

7. The composition according to claim 6, wherein the polypeptide chain comprises a thrombin cleavage site.

8. The composition according to claim 1, wherein the targeting moiety and thrombomodulin domain are chemically cross-linked.

9. The composition according to claim 8, wherein the chemical cross-linkage is via biotin and strepatavidin.

10. The composition according to claim 1, wherein the targeting moiety binds to a cell adhesion molecule exposed on the surface of vascular endothelium.

11. The composition according to claim 10, wherein the cell adhesion molecule is PECAM-1, 1CAM-1, or VCAM-1.

12. The composition according to claim 1, wherein the targeting moiety binds a determinant expressed on the surface of a red blood cell at a density greater than 5,000 copies per red blood cell.

13. The composition according to claim 1, wherein the targeting moiety binds a determinant expressed on the surface of a red blood cell, and said determinant is not a specific site for recognition by host defense cells that clear microscopic objects from the surface of a red blood cell without damage to the red blood cell.

14. The composition according to claim 1, wherein the targeting moiety binds glycophorin A or a glycophorin A associated protein.

15. The composition according to claim 1, wherein the targeting moiety binds an ABO blood group antigen.

16. A pharmaceutical composition comprising a composition of claim 1 and a pharmaceutically acceptable carrier.

17. A method of delivering a thrombomodulin domain to a luminal surface of vascular endothelium comprising delivering a composition according to claim 1 to a blood vessel.

18. A method of delivering a thrombomodulin domain to the surface of a red blood cell comprising delivering a composition according to claim 1 to a blood vessel.

19. A method of treating, inhibiting, or preventing thrombosis, tissue ischemia, acute myocardial infarction (AMI), non-ST segment elevated AMI, deep vein thrombosis, hyperoxic injury, transient ischemic attack (TIA)ischemic stroke, cerebrovascular disease, disseminated intravascular coagulation (DIC), pulmonary embolism, ischemic peripheral vascular disease, inflammation, pulmonary edema, sepsis, acute lung injury (ALI), acute respiratory distress syndrome (ARDS), or aseptic systemic inflammation, comprising administering a composition according claim 1.

20. (canceled)

21. The method according to claim 19, wherein said method is for treating, inhibiting, or preventing thrombosis.
